(19) United States
(12) Patent Application Publication
(10) Pub. No.: US 2026/0263005 A1
(43) Pub. Date: Sep. 10, 2026

(54) Wearable Medical Devices, Applicators Therefor, and Methods of Using the Same

(71) Applicant: Solventum Intellectual Properties Company, Maplewood, MN (US)

(72) Inventors: Audrey A. Sherman, Woodbury, MN (US); Tony J. Kaufman, Rosemount, MN (US); Jeremy K. Larsen, Farmington, MN (US); Kyle C. Picha, Maplewood, MN (US); Brian J. Gates, Osceola, WI (US); Alexander J. Huffman, St. Paul, MN (US); Del R. Lawson, Cottage Grove, MN (US); Daniel J. Rogers, Grant, MN (US); Stephen R. Alexander, Stillwater, MN (US); Dylan T. Cosgrove, Oakdale, MN (US); Gnana Saurya Vankayalapati, St. Paul, MN (US)

(21) Appl. No.: 19/164,688

(22) PCT Filed: Mar. 19, 2024
(86) PCT No.: PCT/IB2024/052662

§ 371 Date: Sep. 12, 2025

Related U.S. Application Data
(60) Provisional application No. 63/491,147, filed on Mar. 20, 2023.

(51) Int. Cl. A61B 5/00

(52) U.S. Cl. CPC A61B 5/685; A61B 5/6802; A61B 5/6832

(57) ABSTRACT

Wearable medical devices having a plurality of microneedles on first rotationally-distinct segment and a plurality of microneedles on a second rotationally-distinct segment are described. Applicators and methods for attaching a wearable medical device to a skin surface are also described.

### BACKGROUND

The industry for wearable medical and/or fitness monitoring devices is growing. People are becoming more interested in monitoring their health and remotely sharing their health data with practitioners or emergency personnel. Many current devices that monitor parameters such as heart rate, blood pressure, oxygen saturation, and the like, are in the form of wearable jewelry, e.g., watches, bracelets, rings, chest straps, etc. However, not all parameters can be measured in this manner, nor are these wearables discreet. For example, continuous glucose monitoring, via a device adhered to the skin, is gaining popularity amongst diabetics and even amongst people who are following low-sugar diets. However, the adhesives required to wear such devices often cause skin injuries and infection, especially in elderly users. Adhesives are also known to cause allergic reactions in some individuals, which can be severe enough to preclude some patients from using the devices.

What is needed are ways in which to secure monitoring devices to skin without requiring the use of adhesives.

### SUMMARY

In one embodiment, a wearable medical device is described. The wearable medical device includes a base having a first rotationally-distinct segment and a second rotationally-distinct segment. The second rotationally-distinct segment at least partly surrounds the first rotationally-distinct segment. The wearable medical device further includes at least one communication member in communication with the first rotationally-distinct segment and the second rotationally-distinct segment, a plurality of first microneedles located on the first rotationally-distinct segment, and a plurality of second microneedles located on the second rotationally-distinct segment.

In one embodiment, an applicator for attaching a wearable medical device of the present disclosure to a skin surface is described. The applicator includes a loading actuator configured to counter-rotationally load the first rotationally-distinct segment and the second rotationally-distinct segment, a retaining element to retain the wearable medical device in a counter-rotationally loaded configuration, and a mechanism for releasing the wearable medical device from the counter-rotationally loaded configuration.

In one embodiment, a method for attaching a wearable medical device of the present disclosure to a skin surface is described. The method includes rotating a first rotationally-distinct segment and rotating the second rotationally-distinct segment such that the wearable medical device is in a counter-rotationally loaded configuration, contacting the wearable medical device in the counter-rotationally loaded configuration to the skin surface, and releasing the wearable medical device from the counter-rotationally loaded configuration such that a plurality of first microneedles and a plurality of second microneedles are driven into the skin surface.

In one embodiment, a method for monitoring a biological signal is described. The method includes detecting a biological signal with a monitoring device that is secured to a wearable medical device of the present disclosure, the wearable medical device being attached to a skin surface.

In one embodiment, a kit is described. The kit includes a wearable medical device of the present disclosure and a set of instructions for attaching the wearable medical device to a skin surface.

### BRIEF DESCRIPTION OF DRAWINGS

The present application may be more completely understood in consideration of the following detailed description of various embodiments of the disclosure in connection with the accompanying drawings.

FIG. 1A is a bottom side view of a wearable medical device of the present disclosure.

FIG. 1B is a top view of the wearable medical device of FIG. 1A.

FIG. 2A is a top side view of a wearable medical device of the present disclosure.

FIG. 2B is a top view of the wearable medical device of FIG. 2A.

FIG. 3A is a bottom side view of a wearable medical device of the present disclosure.

FIG. 3B is a top view of the wearable medical device of FIG. 2A.

FIG. 4 shows a side view of a microneedle of the present disclosure and an elevated angle measurement.

FIG. 5 shows a top view of microneedles of the present disclosure and an orientation angle measurement.

FIG. 6A shows an applicator with an inserted wearable medical device of the present disclosure.

FIG. 6B shows an applicator with a wearable medical device in a counter-rotationally loaded configuration.

FIG. 7A is a top view of the wearable medical device in an unloaded configuration having mechanical actuators.

FIG. 7B is a top view of the wearable medical device in a loaded configuration having mechanical actuators.

Reference is made to the accompanying drawings in the description below. Various embodiments in which the disclosure may be practiced are provided by way of illustration. It is to be understood that structural changes may be made without departing from the scope of the present disclosure. The figures are not necessarily to scale. Like numbers used within the figures refer to like components (e.g., 102, 202, 302, etc.; 110, 210, 310, etc.; and the like).

### DETAILED DESCRIPTION

The present disclosure describes wearable medical devices that can be secured to the skin via microneedles. The wearable medical devices make use of opposing forces between rotational segments to not only drive said microneedles into the skin, but to also secure said microneedles within the skin. Wearable medical devices that are attached to the skin via microneedles are much more resistant to accidental removal and have extended wear time relative to comparable devices adhered to skin via adhesives. Moreover, wearable medical devices of the present disclosure are painless to attach, painless to wear, and do not cause skin injury or adverse reactions that are often accompanied by adhesives. Additionally, the wearable medical devices of the present disclosure allow for air flow beneath the device to prevent bacterial growth from moisture buildup and further allow for cleaning.

While the wearable medical devices may include a permanent monitoring device thereon, it is largely intended that the wearable medical devices of the present disclosure serve as a baseplate for securing removable monitoring devices thereto. Users may enjoy the versatility of a modular system.

### Definitions

As used herein, the term “about” means±10 percent of a given value. For example, about 10 means 9 to 11.

As used herein, the term “adhesive” as used herein refers to polymeric compositions that adhere together two adherends. Examples of adhesives are pressure sensitive adhesives and gel adhesives.

As used herein, the term “actuating guide” refers to a feature on or within a component of an applicator that is complementary to an applicator guide within a wearable medical. The mating of an actuating guide and an applicator guide, under means of rotation within the applicator, is effective to rotate a first and/or second rotationally-distinct segment within the wearable medical device.

As used herein, the term “applicator guide” or “applicating guide” refers to a feature on or within a component of a wearable medical device that is complementary to an actuating guide within an applicator. The mating of an applicator guide and an actuating guide, under means of rotation within the applicator, is effective to rotate a first and/or second rotationally-distinct segment within the wearable medical device.

As used herein, the term “barb” describes a feature on a microneedle body that extends outward at some angle from the microneedle body. A barbed needle may be harder to remove from a skin surface than a non-barbed needle. Likewise, a barbed needle may prevent complete penetration compared to a non-barbed needle. Barbed needles may increase attachment, thereby extending wear times. Barbed needles may also aid in achieving desired gaps between the wearable medical device and the skin surface.

As used herein, “center” means a point in which two perpendicular planes meet and each of the areas in the respective four quadrants are equal. For example, the center of a microneedle base is the center of the area in contact with the respective rotationally-distinct segment.

As used herein, the term “communication member” refers to a substance connecting the first rotationally-distinct segment and the second rotationally-distinct segment, but said substance does not prevent independent rotation of the first rotationally-distinct segment and the second rotationally-distinct segment. As used herein, the term “tensioning communication member” refers to an article connecting the first rotationally-distinct segment and the second rotationally-distinct segment, wherein potential energy is stored within the article that is deformed upon rotating the first and second rotationally-distinct segment and converted to kinetic energy when the article is allowed to return at least partly to its original state. As used herein, “rolling communication member” refers to a rotating article located at least partly between the first and second rotationally-distinct segments, wherein upon rotating the first and second rotationally-distinct segment, the rotating article thus rotates accordingly.

As used herein, the term “counter-rotationally” is used to describe the way the first rotationally-distinct segment and the second rotationally-distinct segment are rotated relative to one another. One segment is rotated clockwise, and the other segment is rotated counterclockwise.

As used herein, “flexible” describes articles that may be stretched, bent, compressed, or otherwise twisted under force, yet returns at least partly to an unstretched, unbent, uncompressed, or untwisted state when said force is removed.

As used herein, the term “microneedles” refers to microstructural protrusions with pointed tips configured to penetrate skin.

As used herein, “rotation” means to move some degree around an axis of rotation.

As used herein, the phrase “rotationally-distinct” describes a component that may be rotated independent another component. For example, two rotationally-distinct components that are otherwise connected are capable of be rotated to some degree in opposing directions.

### FIGURE DESCRIPTION

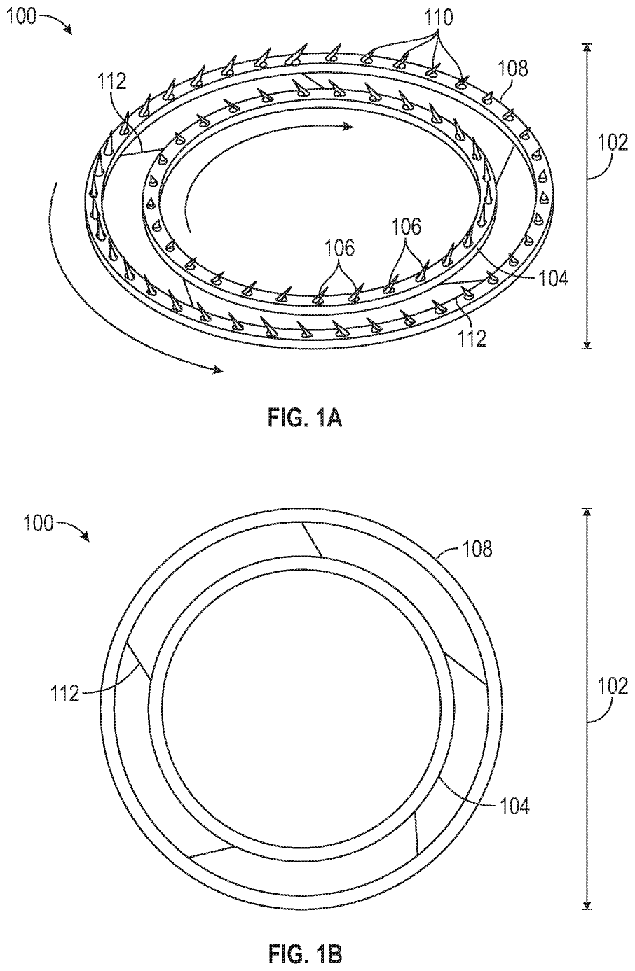

FIG. 1A is a bottom view of a wearable medical device 100 of the present disclosure, illustrating a first major surface of base 102. Wearable medical device 100 includes a base 102 having a first rotationally-distinct segment 104 with a plurality of first microneedles 106 thereon, and a second rotationally-distinct segment 108 with a plurality of second microneedles 110 thereon. First rotationally-distinct segment 104 and second rotationally-distinct segment 108 are shown in the shape of concentric cylindrical rings that are connected by (tensioning) communication members 112 (shown here as flexible rods or bands). Communication members 112 are depicted connecting first rotationally-distinct segment 104 and second rotationally-distinct segment 108 in a non-radial fashion. During application, a loading actuator (not shown) rotates first rotationally-distinct segment 104 in a direction counter to first microneedles 106 tips (shown here clockwise) and rotates second rotationally-distinct segment 108 counter to second microneedle 110 tips (shown here counterclockwise) thereby contracting communication members 112. Communication members 112 stretch upon rotating the first rotationally-distinct segment and second rotationally-distinct segment. One may easily envision alternate arrangements in which communication members 112 are otherwise bent. A retaining element (not shown) holds the respective rotationally-distinct segments 104/108 in a counter-rotationally loaded configuration. Upon contact with the skin, a retention element (not shown) may be disengaged to release wearable medical device 100, in which the directionally-opposed first and second microneedles 106/110 are each driven into the skin surface by way of (tensioning) communication members 112 returning at least partly to a de-tensioned state.

FIG. 1B is a top view of the wearable medical device 100 of FIG. 1A, illustrating a second major surface of base 102. First rotationally-distinct segment 104 and second rotationally-distinct segment 108 are shown in the shape of concentric cylindrical rings that are connected by communication members 112.

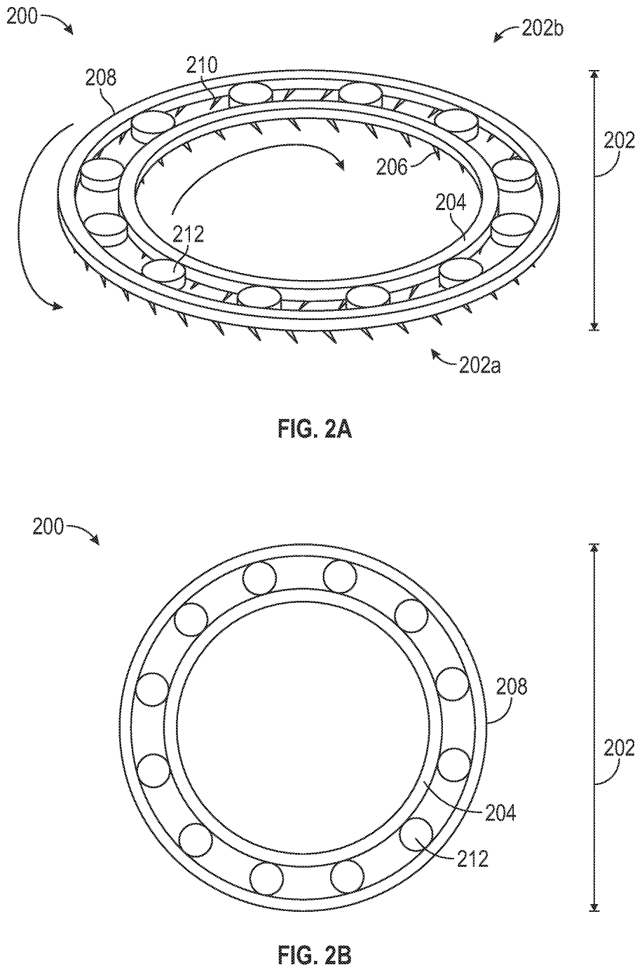

FIG. 2A is a top side view of a wearable medical device 200 of the present disclosure, illustrates a first major surface 202a and a second major surface 202b of base 202. Wearable medical device 200 includes a base 202 having a first rotationally-distinct segment 204 with a plurality of first microneedles 206 thereon, and a second rotationally-distinct segment 208 with a plurality of second microneedles 210 thereon. First rotationally-distinct segment 204 and second rotationally-distinct segment 208 are shown in the shape of concentric cylindrical rings that are in mechanical communication by (rolling) communication members 212 (shown here as rolling discs). During application, a drive actuator (not shown) rotates first rotationally-distinct segment 204 in a direction aligned with first microneedles 206 tips (shown here clockwise) and rotates second rotationally-distinct segment 208 in a direction aligned with second microneedle 210 tips (shown here counterclockwise) thereby rolling communication members 212. Upon contact with the skin, said drive actuator drives the first and second microneedles 206/210 into the skin surface.

FIG. 2B is a top view of the wearable medical device 200 of FIG. 2A, illustrating a second major surface of base 202. First rotationally-distinct segment 204 and second rotationally-distinct segment 208 are shown in the shape of concentric cylindrical rings that are in mechanical communication by (rolling) communication members 212.

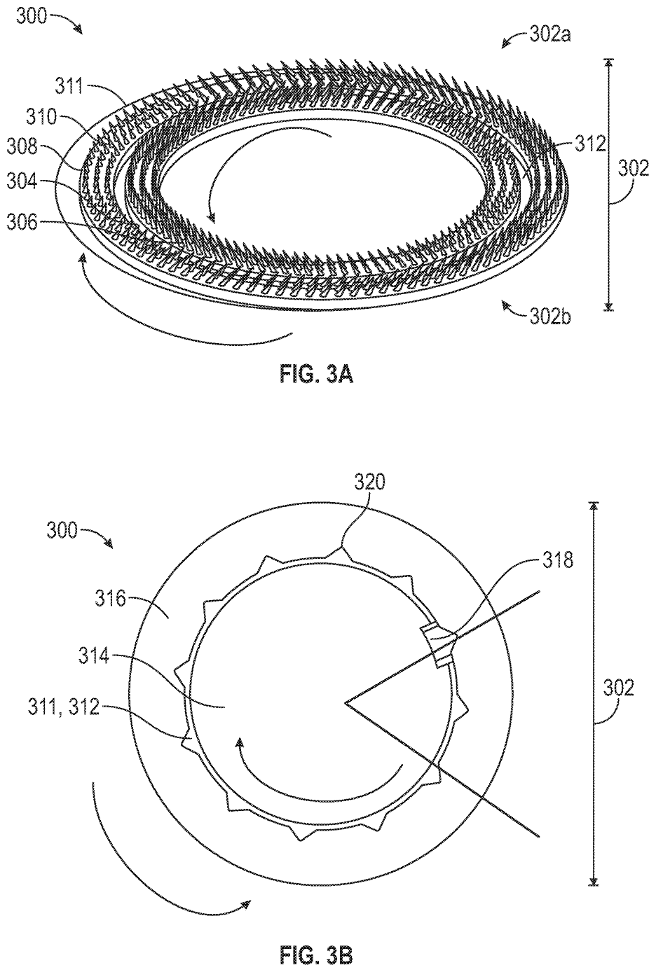

FIG. 3A is a bottom side view of a wearable medical device 300, illustrating a first major surface 302a and a second major surface 302b of a base 302. Wearable medical device 300 includes base 302 having a first rotationally-distinct segment 304 with a plurality of first microneedles 306 thereon, and a second rotationally-distinct segment 308 with a plurality of second microneedles 310 thereon. First rotationally-distinct segment 304 and second rotationally-distinct segment 308 are shown in the shape of concentric cylindrical rings, and the respective microneedles 306/310 are flushed in three rows each. Wearable medical device 300 further includes a flexible membrane 311 in contact with second major surface 302b. Flexible membrane 311 is adhered to first rotationally-distinct segment 304 and a second rotationally-distinct segment 308, and acts as a (tensioning) communication member 312 therebetween. During application, a loading actuator (not shown) rotates first rotationally-distinct segment 304 in a direction counter to first microneedles 306 tips (shown here counterclockwise) and rotates second rotationally-distinct segment 308 counter to second microneedle 310 tips (shown here clockwise) thereby stretching flexible membrane 311/communication member 312 therebetween. A retaining element (not shown) holds the respective rotationally-distinct segments 304/308 in a counter-rotationally loaded configuration. Upon contact with the skin, a retention element (not shown) may be disengaged to release wearable medical device 300, in which the opposed first and second microneedles 306/310 are driven into the skin surface by way of flexible membrane 311/communication member 312 returning at least partly to a de-tensioned state.

FIG. 3B is a top view of the wearable medical device 300 in FIG. 1A, illustrating the second major surface of base 302. A portion of flexible membrane 311/communication member 312 can be viewed between an inner placement backing 314, which overlays first rotationally-distinct segment (not shown) and an outer placement backing 316, which overlays second rotationally-distinct segment (not shown). Inner placement backing 314 is shown to have an inner applicating guide 318 and outer placement backing 316 is shown to have outer applicating guide 320. During application, a loading actuator (not shown) rotates the first rotationally-distinct segment (not shown) in a direction (shown here clockwise) by way of communication with inner placement backing 314/inner applicating guide 318 and rotates the second rotationally-distinct segment (not shown) in a counter direction (shown here counterclockwise) by way of communication with outer placement backing 316/outer applicating guide 320. In effect, the flexible membrane 311/communication member 312 is stretched or otherwise twisted. A retaining element (not shown) holds the respective rotationally-distinct segments in a counter-rotationally loaded configuration. Upon contact with the skin, a retention element (not shown) may be disengaged to release wearable medical device 300, in which the first and second microneedles (not shown) are driven into the skin surface by way of flexible membrane 311/communication member 312 returning at least partly to a de-tensioned state.

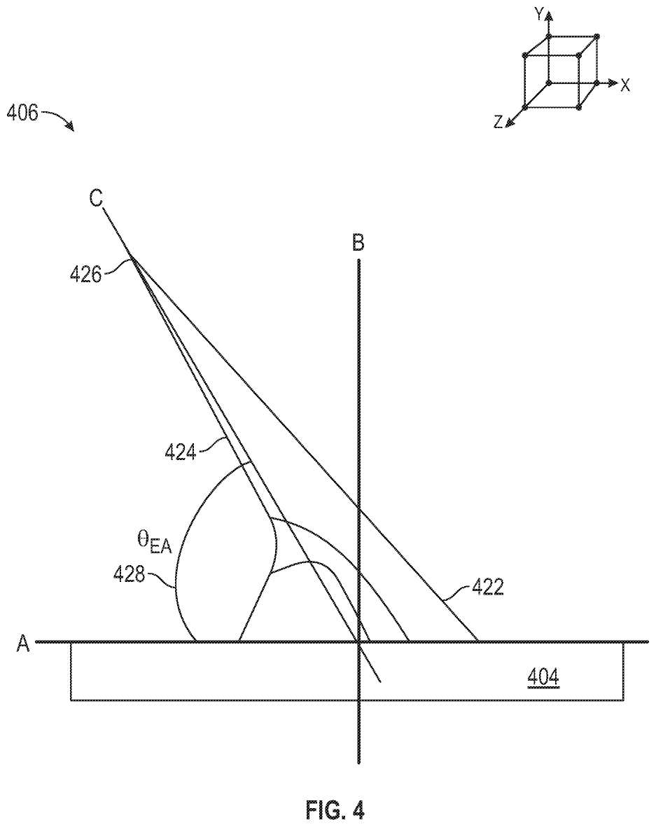

FIG. 4 illustrates a side view of an example first microneedle 406 (or second microneedle) of the present disclosure. First microneedle 406 is shown with a microneedle base 422 that is in contact with first rotationally-distinct segment 404. Microneedle base 422 extends into a microneedle body 424 and terminates at a microneedle tip 426. First microneedle 406 is shown at an elevation angle 428 (“θ_{EA}”). Elevation angle 428 is measured from a plane A that is parallel to the surface in which first microneedle 406 contacts first rotationally-distinct segment 404 to microneedle tip 426 (see plane C), relative to a plane B that passes through the center of microneedle base 422—plane A and plane B are perpendicular to each other, i.e., 90°.

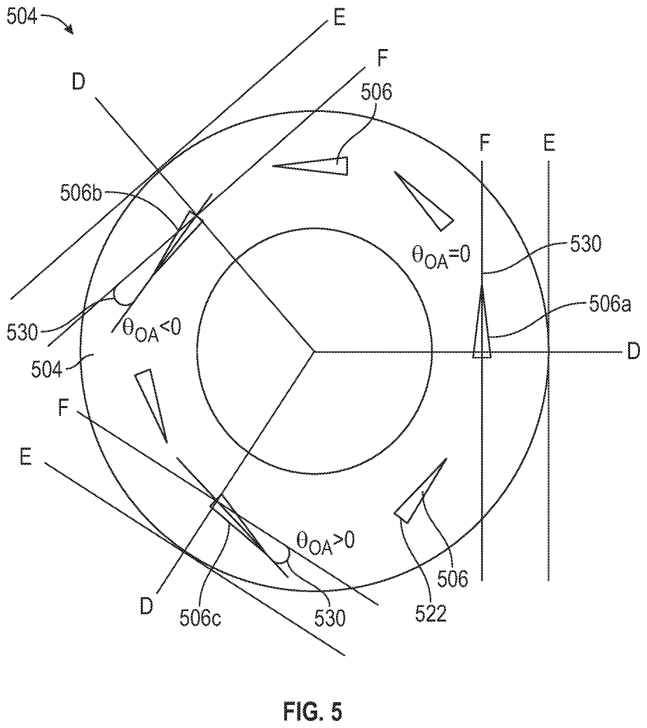

FIG. 5 illustrates a top view of a first rotationally-distinct segment 504 having a plurality of first microneedles 506 arranged thereon (or second microneedles on a second rotationally-distinct segment) of the present disclosure. Each of the plurality of first microneedles 506 are independently arranged at an orientation angle 530 (“θ_{OA}”). Orientation angles 530 are measured with respect to a radial plane D and a tangential plane E (i.e., tangential plane E is tangent, i.e., 90°, to radial plane D). Plane F is parallel to plane E and is simply for visual aid. Each radial plane D passes through the center of a microneedle base 522 (see plane B in FIG. 4, i.e., radial plane D is in the z direction perpendicular to plane B; tangential plane E and plane A is in the x direction). A first microneedle 506a that is aligned with tangential planes E and F has an orientation angle 530 of 0°, i.e., θ_{OA}=0°. A first microneedle 506b that is angled toward the axis of rotation is characterized by an orientation angle 530 of less than 0° by some measurable degree, i.e., θ_{OA}<0°, e.g., −10°. A first microneedle 506c that is angled away from the axis of rotation is characterized by an orientation angle 530 of greater than 0° by some measurable degree, i.e., θ_{OA}>0°, e.g., 10°. The descriptions need not be limited to circular constructions.

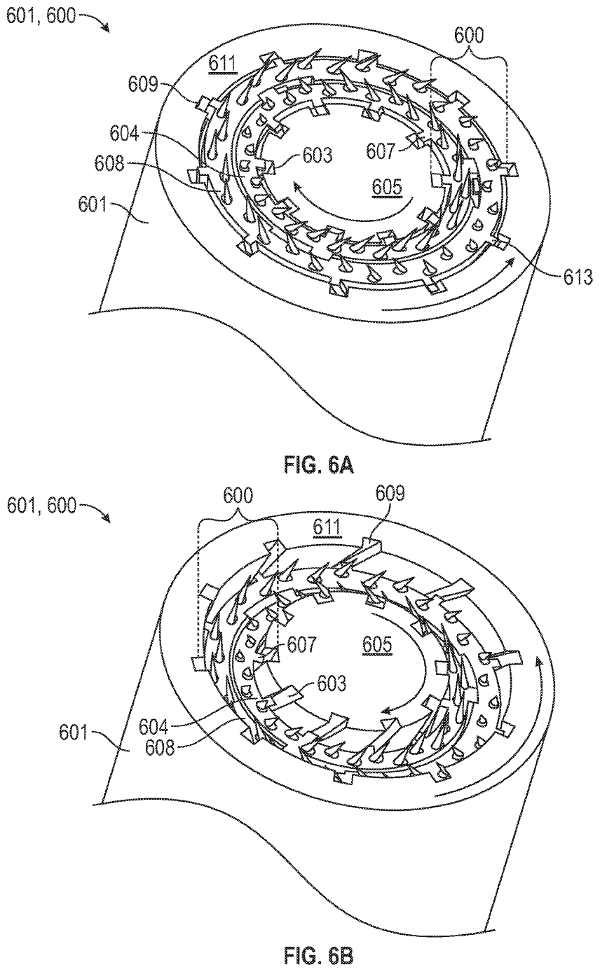

FIG. 6A illustrates an example applicator 601 with an inserted wearable medical device 600 in an unloaded configuration. Applicator 601 is shown to include first segment actuating guides 603 within an interior wall 605 that mate with first applicator guides 607 located on first rotationally-distinct segment 604. Applicator 601 is further shown to include second segment actuating guides 609 within an exterior wall 611 that mate with second applicator guides 613 located on second rotationally-distinct segment 608.

FIG. 6B illustrates the example applicator 601 having wearable medical device 600 in a counter-rotationally loaded configuration. First rotationally-distinct segment 604 has been rotated clockwise and second rotationally-distinct segment 608 has been rotated counterclockwise. Applicator 601 retains wearable medical device 600 in this counter-rotationally loaded configuration (retaining element not shown) until applicator 601 is contacted to a skin surface. Upon disengaging a retention element (not shown), wearable medical device 600 is released from the loaded configuration and the plurality of microneedles on each segment are driven into the skin surface.

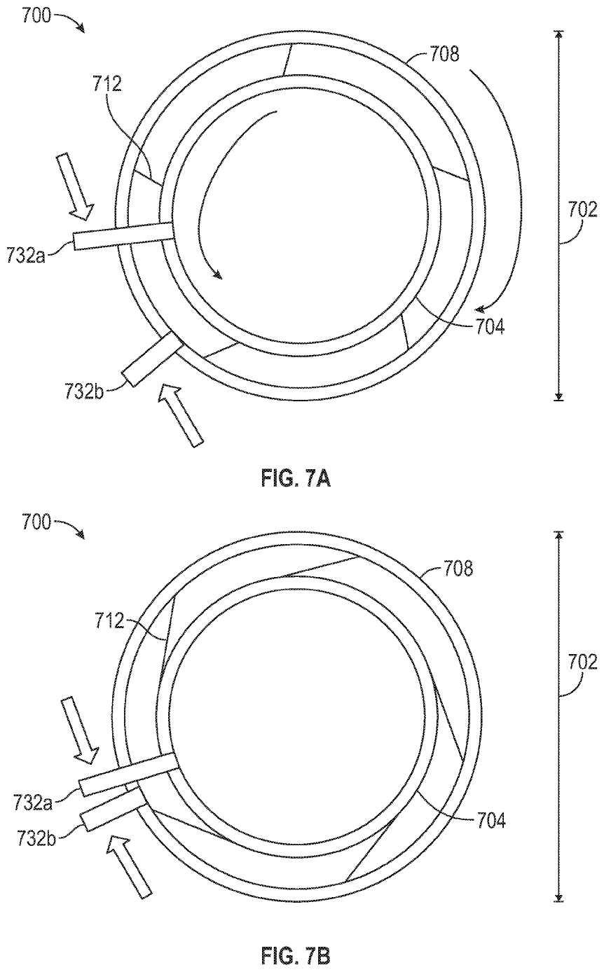

FIG. 7A illustrates a top view (second major surface) of a wearable medical device 700 having a set of mechanical actuators 732a/732b, wherein the medical device is shown in an unloaded configuration. Wearable medical device 700 includes a base 702 having a first rotationally-distinct segment 704, a second rotationally-distinct segment 708, and communication members 712. First mechanical actuator 732a is in communication with first rotationally-distinct segment 704, and second mechanical actuator 732b is in communication with second rotationally-distinct segment 708. Upon squeezing the mechanical actuators 732a/732b together, first rotationally-distinct segment 704 is rotated counterclockwise and second rotationally-distinct segment 708 is rotated clockwise. Mechanical actuators 732a/732b may be used to attach a wearable medical device to a skin surface or remove a wearable medical device from a skin surface.

FIG. 7B illustrates a top view of a wearable medical device 700 having a set of mechanical actuators 732a/732b, wherein the medical device is shown in a loaded configuration. Communication members 712 are shown to be stretched in comparison with the unloaded configuration of FIG. 7A.

### Wearable Medical Devices

In various embodiments, a wearable medical device is described. The wearable medical device may include a base having a first rotationally-distinct segment and a second rotationally-distinct segment. The second rotationally-distinct segment may at least partly surround the first rotationally-distinct segment. At least one communication member may be in communication with the first rotationally-distinct segment and the second rotationally-distinct segment. The wearable medical device may further include a plurality of first microneedles located on the first rotationally-distinct segment and a plurality of second microneedles located on the second rotationally-distinct segment.

Further details and further features of wearable medical devices are described below. It is to be understood that the details and features described below may be incorporated alone or in combination unless stated otherwise.

### Base

The base, and all components within the base, may be characterized by a first major surface and a second major surface. The first major surface is deemed to be the skin-contacting surface, whereas the second major surface is opposite the first major surface and does not contact the skin when the wearable medical device is in use. As such, all first and second microneedles described herein are located on the first major surface of the base.

In some embodiments, the base may further include one or more applicator guide for mating with an applicator, the applicator guide being configured rotate the first rotationally-distinct segment and the second rotationally-distinct segment. For example, an applicator guide may be in the form of one or more notch, protrusion, pin, pin hole, or the like, wherein said applicator guide may be complementary to an actuating guide within an applicator. Applicator guides may be located on the second major surface, along a periphery (minor surfaces), or a combination thereof.

In some embodiments, the base may further include one or more monitoring device securement feature for attaching a monitoring device to the wearable medical device. Example monitor device securement features may include clips, hooks, latches, brackets a threaded component for mating with a threaded monitoring device, an adhesive, or a combination thereof, or the like. Monitoring device securement features may be located on the second major surface, along a periphery (minor surfaces), or a combination thereof.

In some embodiments, the base may further include a first mechanical actuator in communication with the first rotationally-distinct segment and a second mechanical actuator in communication with the second rotationally-distinct segment. FIGS. 7A and 7B illustrate an example mechanical actuation of the wearable medical device absent the use of an applicator described herein. While FIGS. 7A and 7B demonstrate counter-rotationally loading a wearable medical device (i.e., the mechanical actuators pushed together), the opposite may also be envisioned. For example, a wearable medical device having rolling communication members (e.g., FIG. 2A) may include mechanical actuators that may be used to drive (i.e., the mechanical actuators pushed apart) the plurality of microneedles into the skin surface.

In some embodiments, mechanical actuators may be used to apply and/or remove a wearable medical device from a skin surface, with or without an applicator described herein. While mechanical actuators are not necessary to employ an applicator described herein, an applicator may be configured to actuate said mechanical actuators. In other words, any such mechanical actuators may be considered an “applicator guide” as used herein, when in combination with an applicator.

In some embodiments, the base may further include a flexible membrane adhered to or otherwise connected to the second major surface and extending at least from the first rotationally-distinct segment to the second rotationally-distinct segment such that the first rotationally-distinct segment may be in communication (i.e., communication member) with the second rotationally-distinct segment. In some embodiments, the flexible membrane may span the entirety of the second major surface of the base. In some embodiments, the flexible membrane may extend beyond the periphery of the base. A base having a flexible membrane extending beyond the periphery of the base may further include an adhesive thereon may serve as a secondary skin attachment modality.

In some embodiments, a flexible membrane may be comprised of material such as a woven fabric (e.g., cotton, rayon, polyvinyl chloride, polyethylene, or polyurethane), latex, or the like. In some embodiments, the flexible membrane may be breathable and waterproof.

In some embodiments, the flexible membrane may further include an adhesive on one or more surface. In some embodiments, a suitable adhesive may be comprised of an acrylate, methacrylate, epoxy diacrylates, or the like. An adhesive may be located on a surface that is to contact skin surface upon application and therefore serve as a secondary means to secure the wearable medical device to the skin. An adhesive may be located on a surface that is to be opposite a skin surface upon application and may serve as means to attach a placement backing and/or a monitoring device (i.e., a monitoring device securement feature). In some embodiments, a flexible membrane may be in the form of a double-sided tape.

In many embodiments, a flexible membrane may be transmissible to light. In many embodiments, a flexible membrane may be constructed from material that is readily puncturable (e.g., by a needle). In other embodiments, a flexible membrane may include an area void of material for passage of a needle (e.g., a needle extending from a mounted glucose monitor device), light (e.g., transmitted from a mounted oximeter device), an electrode, or some other skin-contacting or penetrating probe.

In many embodiments, a base may further include a flexible membrane described herein and one or more placement backing. The one or more placement backing may be reversibly or irreversibly adhered to the flexible membrane with an adhesive or may otherwise be sewn thereon. A placement backing may include applicating guides configured to mate with a loading actuator within an applicator. In some embodiments, a placement backing may include an inner placement backing configured to rotate the first rotationally-distinct segment (e.g., by way of inner applicating guides) and an outer placement backing, at least partly surrounding the inner placement backing, configured to rotate the second rotationally-distinct segment (e.g., by way of outer applicating guides).

### Rotationally-Distinct Segments

In many embodiments, the first and second rotationally-distinct segments may be arranged such that a common axis of rotation is shared. While individual axes of rotation may be envisioned and are intended to be encompassed by the scope of the disclosure, a shared axis of rotation is the simplest and most concise construction.

In many embodiments, the first and second rotationally-distinct segment are configured to be rotated in opposite directions (i.e., clockwise, and counterclockwise relative to one another), wherein said rotations induce a stress within a communication member that is in communication with each. Said stress may be in the form of stretching, compressing, twisting, bending, coiling, rolling, rotating, or the like. An applicator of the present disclosure, or other applicator means, may be configured to secure the first and second rotationally-distinct segments in the rotated states and withstand the potential energy within the stressed communication member. The kinetic energy afforded by the release of stress within the communication member is effective to de-rotate the rotationally-distinct segments such that the microneedles thereon may be driven into the skin under some force.

The first and second rotationally-distinct segments may independently be of any size and shape so long as neither segment impedes the rotation of the other. Example shapes include full- or semi-cylinders, elliptic cylinders, conical frustums, rectangles, squares, frustrum, or the like; the shapes may be either solid or annular (i.e., ring-like). An annular-shaped first rotationally-distinct segment may allow for passage of light from a mounted monitoring device, or otherwise physical contact between the skin surface and a mounted monitoring device. In some embodiments, the first and second rotationally-distinct segments may each be cylindrical rings (i.e., washer-like) and arranged in a concentric manner. In other embodiments, the first rotationally-distinct segment may be a solid cylinder and the second rotationally-distinct segment may be a cylindrical ring, arranged in a concentric manner. Some shapes may be better suited for different applications, e.g., to accommodate different areas of the body, to accommodate differently-shaped monitoring devices, or the like.

In many embodiments, the first and the second rotationally-distinct segments are arranged such that at least one major surface of each of the segments are flush with each other. Any embodiment in which the first and the second rotationally-distinct segment do not include at least one major surface of each of the segments being flush with each other, the wearable medical device will require the first microneedles and the second microneedles to not be equal in length so as for each set of microneedles to be able to contact the skin.

In some embodiments the first and second rotationally-distinct segments may independently be characterized by a greatest length and greatest width of about 5 mm to about 75 mm. For example, the greatest lengths and/or greatest widths may be selected from, in mm, about 5, 10, 15, 20, 25, 30, 35, 40, 45, 50, 55, 60, 65, 70, or 75, or a value within a range between any of the preceding values, e.g., between about 25 and about 40, or the like.

In some embodiments, the first and second rotationally-distinct segment may independently be characterized by an average thickness of about 1 mm to about 10 mm. For example, the average thicknesses may be selected from, in mm, about 1, 1.5, 2, 2.5, 3, 3.5, 4, 4.5, 5, 5.5, 6, 6.5, 7, 7.5, 8, 8.5, 9, 9.5, or 10, of a value within a range between any of the preceding values, e.g., between about 3 and about 8, or the like.

In many embodiments, the first and second rotationally-distinct segments may each include at least 10 microneedles thereon. In some embodiments, the first and second rotationally-distinct segment may each independently include 10-500 microneedles thereon. For example, the first and second rotationally-distinct segment may each independently include a number of microneedles of 10, 20, 30, 40, 50, 60, 70, 80, 90, 100, 125, 150, 175, 200, 225, 250, 275, 300, 325, 350, 375, 400, 425, 450, 475, or 500, or a value within a range between any of the preceding values, e.g., between about 50 and about 100, or the like. The number of needles in each of the rotationally-distinct segments may be selected according to various factors such intended device placement, skin type, level of activity of user, intended wear time, or the like.

In some embodiments, the first rotationally-distinct segment may include one or more first applicator guide configured to mate with one or more actuator guide within an applicator described herein. The one or more first applicator guide may be located on an inner perimeter (minor surface) of the first rotationally-distinct segment. In some embodiments, the second rotationally-distinct segment may include one or more second applicator guide configured to mate with one or more actuator guide within an applicator described herein. The one or more second applicator guide may be located on an outer perimeter (minor surface) of the second rotationally-distinct segment. In some embodiments, the first and second applicator guide(s) may independently be in the form of notches, protrusions, pins, pin holes, or the like.

In some embodiments, the first and second rotationally-distinct segments may be comprised of a material selected from a metal, a plastic, or a combination thereof.

In some embodiments, the wearable medical device may have only two rotationally-distinct segments. In other embodiments, the wearable medical device may have more than two rotationally-distinct segments, wherein any additional rotationally-distinct segment may be characterized in a similar manner as to any rotationally-distinct segment described herein.

### Communication Member

In some embodiments, a communication member may be a tensioning communication member selected from flexible rods or bands, springs, a flexible membrane (described above), a combination thereof, or the like. In other embodiments, a communication member may be a rolling communication member such as rolling discs.

In some embodiments, a communication member may be in the form of a flexible rod, a flexible band, or a spring.

In many embodiments, a communication member may at least partly connect the first rotationally-distinct segment and the second rotationally-distinct segment via a minor surface (e.g., an interior wall or exterior wall of rotationally-distinct segments in the shape of a ring). In some embodiments, a communication member may at least partly connect the first rotationally-distinct segment and the second rotationally-distinct segment via a major surface (e.g., the second major surface opposite the first major surface having the microneedles).

In some embodiments, a wearable medical device may include one or more communication member in the form of a flexible rod or band extending from an exterior wall of a first rotationally-distinct segment that is in the shape of a ring and an interior wall of second rotationally-distinct segment that is in the shape of a ring. In some embodiments, the flexible rod or band may extend radially (i.e., parallel to a radius) between the first and second rotationally-distinct segments. In other embodiments, the flexible rod or band may extend non-radially (e.g., at an angle relative to a radial plane) between the first and second rotationally-distinct segments. A non-radially arrangement may be measured according to one end of a communication member taken to be on a radial plane and the other end of the communication measured at an angle of about 1°-45° relative to the radial plane, e.g., an angle (°) of 1, 2, 5, 8, 10, 12, 15, 18, 20, 22, 25, 28, 30, 32, 35, 38, 40, 42, or 45 degrees, or a value within a range between any of the preceding values, e.g., between about 20 and about 40. A flexible rod or band in a non-radially arrangement may be positioned in one of two orientations, i.e., / or \, and depending upon the rotation directions of the rotationally-distinct segments, said flexible rod or band may be stretched or bent.

In some embodiments, the type of communication member and number of communication members present in a wearable medical device of the present disclosure may be selected according to a desired kinetic energy for driving the rotationally-distinct segments. For example, wearable medical devices may be tailored to the type of skin surface that the device is to be applied, which may require more force or less force to install the wearable medical device adequately or safely into the skin surface.

In some embodiments, the wearable medical device may include 1-20 communication members, e.g., 1, 2, 3, 4, 5, 6, 7, 8, 9, 10, 12, 15, 18, or 20, or a value within a range between any of the preceding values, e.g., between 2 and 6, or the like. In some embodiments, each of the communication members are the same type. In other embodiments, a mixture of communication members may be present within the wearable medical device.

### Microneedles

In many embodiments, the first and second microneedles may be arranged in a circular or semi-circular array extending around an axis of rotation, regardless of the shape in which the first and second rotationally-distinct segment. In many embodiments, the plurality of first microneedles may be arranged in one or more row along the first rotationally-distinct segment. Likewise, the plurality of second microneedles may be arranged in one or more row along the second rotationally-distinct segment in a circular path. In some embodiments, rows may be flush with adjacent rows or may be staggard. In some embodiments, each of the plurality of first and second microneedles may be arranged in 1-5 rows, e.g., 1, 2, 3, 4, or 5, e.g., 2-3 rows.

In some embodiments, the plurality of first and second microneedles may be arranged in rows and each of the microneedles may be independently separated from one another by a distance about 1 mm to about 10 mm. For example, any of the microneedles may be separated at a distance, in mm, of about 1, 1.5, 2, 2.5, 3, 3.5, 4, 4.5, 5, 5.5, 6, 6.5, 7, 7.5, 8, 8.5, 9, 9.5, or 10, or a value within a range between any of the preceding values, e.g., between about 4 and about 6, or the like.

In embodiments having more than one row, the rows may be independently separated by a distance about 5 mm to about 10 mm. For example, rows may independently be separated by a distance, in mm, of about 5.5, 6, 6.5, 7, 7.5, 8, 8.5, 9, 9.5, or 10, or a value within a range between any of the preceding values, e.g., between about 6 and about 8, or the like.

In some embodiments, each of the first microneedles and the second microneedles may independently be characterized by an elevation angle of about 40° to about 80° relative to a plane from which the microneedle is attached (i.e., the respective rotationally-distinct segment). For example, the first and second microneedles may be independently characterized by an elevation angle (°) of about 40, 42, 45, 48, 50, 52, 55, 58, 60, 62, 65, 68, 70, 72, 75, 78, or 80, or a value within a range between any of the preceding values, e.g., between about 45 and about 50, or the like. For reference, a microneedle that is perpendicular to the parallel plane passing through the respective rotationally-distinct segment is characterized by an elevation angle of 90°. Elevation angles that are not within the ranges above are still in scope of the present disclosure; however, it is believed that the above ranges may offer benefit to a user in terms of pain mitigation, skin health maintenance, and longer wear times. Obviously, any elevation angle may be measured as an acute angle or an obtuse angle depending upon the point of reference. Thus, the elevation angles described above may be considered as their obtuse counterparts (i.e., about 140° to about 100°, respectively, and everything therebetween). An elevation angle is measured from a parallel plane passing through the respective rotationally-distinct segment to a center of microneedle tip in reference to a plane that is perpendicular to said parallel plane that passes through the center of a microneedle base.

In many embodiments, each of the first microneedles may be characterized by the same elevation angle. In other embodiments, at least a portion of the first microneedles may be characterized by one elevation angle, and at least another portion of the first microneedles may be characterized by another elevation angle. In some cases, a mixture of elevation angles may be beneficial for tuning the wearable medical device for an intended area of the body in which it is to be worn. In many embodiments, each of the second microneedles may be characterized by the same elevation angle. Likewise, in other embodiments, at least a portion of the second microneedles may be characterized by one elevation angle, and at least another portion of the second microneedles may be characterized by another elevation angle. In some embodiments, each of the first and second microneedles may be characterized by the same elevation angle, or some portion of either the first or second microneedles may be characterized by a different elevation angle.

In embodiments having at least a portion of first microneedles characterized by an elevation angle that is not 90°, the first microneedles that are at an elevated angled must all point in the same rotational direction (i.e., all tips facing clockwise or counterclockwise). Likewise, embodiments having at least a portion of second microneedles characterized by an elevation angle that is not 90°, the second microneedles that are at an elevated angled must all point in the same rotational direction. Moreover, in embodiments which have both first microneedles and second microneedles that are characterized by an elevation angle≠90°, the first microneedles at an elevated angle may be situated in the opposite rotational direction relative to the second microneedles at an elevated angle. In other words, each first microneedle characterized by an elevation angle≠90° (e.g., 40° to 80°) may be oriented such that the first microneedle tips face one rotational direction, and each second microneedle characterized by an elevation angle≠90° (e.g., 40° to 80°) may be oriented such that the second microneedle tips face a rotational direction opposite to that of the first microneedle tips. A shared axis of rotation is implied when referring to opposing rotational directions.

In some embodiments, each of the first and second microneedles that are characterized by an elevation angle≠90° may be independently arranged at an orientation angle of about −25° to about 0° (aligned with tangent) or about 0° (aligned with tangent) to about 25° relative to the tangent of a rotation vector (i.e., regarding the rotation of the respective rotationally-distinct segment). Negative orientation angle values indicate the needle is pointed toward the axis of rotation, whereas positive orientation angle values indicate the needle is pointed away from the axis of rotation. For example, any given microneedle may be characterized by an orientation angle (°) of about −25, −22, −20, −18, −15, −12, −10, −8, −5, −2, 0, 2, 5, 8, 10, 12, 15, 18, 20, 22, or 25, or a value within a range between any of the preceding values on either side of 0, e.g., between about −15 and about −8, between about 5 and about 12, or the like. In many embodiments, each of the first and second microneedles may be arranged such that the entirety of the needle body is tangentially aligned with a vector of rotation (i.e., orientation angle of 0°) with respect to the rotation of the respective rotationally-distinct segment. An orientation angle is measured from a tangential plane passing through the center of a microneedle base to the center of microneedle tip. In other words, a microneedle that is parallel to the tangent of a rotation vector is characterized by an orientation angle of 0°. Further, for reference, a microneedle characterized by an orientation angle of 90° would be perpendicular to the vector of rotation and in no way be able to puncture the skin surface during operation of the wearable medical device.

In some embodiments, each of the first and second microneedles may independently be characterized by a length of about 0.2 mm to about 3.0 mm. For example, each of the first and second microneedles may be independently characterized by a length, in mm, of about 0.2, 0.5, 0.8, 1.0, 1.2, 1.5, 1.8, 2.0, 2.2, 2.5, 2.8, or 3.0, or a value within a range between any of the preceding values, e.g., between about 0.5 and about 0.8, or the like. The lengths of needles may be selected in view of application needs. For example, shorter needles may be more comfortable for older users or for areas where skin may be thinner.

In some embodiments, each of the first and second microneedles may be independently characterized by a diameter of about 1 μm to about 25 μm. For example, each of the first and second microneedles may be independently characterized by a diameter, in μm, of about 1, 2, 5, 8, 10, 12, 15, 18, 20, 22, or 25, or value within a range between any of the preceding values, e.g., between about 8 and about 12, or the like. In some embodiments, any microneedle described herein may have a uniform diameter or a nonuniform diameter within the ranges above. A nonuniform diameter may be characterized by a diameter than decreases along the microneedle body toward the tip. For example, a nonuniform diameter may decrease in diameter along the microneedle body toward the tip at a rate of about 5-25%, e.g., 5, 8, 10, 12, 15, 18, 20, 22, or 25%, or a value within a range between any of the preceding values, e.g., 10 to about 15, or the like. A nonuniform diameter may also include areas within the microneedle body that may be larger in diameter, or otherwise isolated areas that may be larger in diameter. Such areas of larger diameter may be in the form of a barb. Microneedles having barbs may serve to better anchor the microneedle within the skin surface. Areas of larger diameter may also prevent the entire microneedle from penetrating the skin, effectively leaving an area between the skin surface and the base to allow airflow therebetween and prevent moisture buildup and/or bacterial growth. In some embodiments, at least a portion of the first and/or second microneedles may be characterized by a nonuniform diameter.

In many embodiments, it is desired to leave a space between the skin surface and the wearable medical device such that airflow may prevent moisture buildup and bacteria growth. One way to achieve this is to select microneedles of a certain length and/or certain diameter. In other words, the microneedles may only be inserted some percentage of the way into the skin. For example, the microneedles may be inserted 25-75% of the length into the skin, e.g., 25, 30, 35, 40, 45, 50, 55, 60, 65, 70, or 75% of the length, or a value within a range between any of the preceding values. In some embodiments, the wearable medical device may sit above the skin surface with a gap thickness of about 0.15 mm to about 1 mm, e.g., 0.15, 0.2, 0.25, 0.3, 0.35, 0.4, 0.45, 0.5, 0.55, 0.60, 0.65, 0.7, 0.75, 0.8, 0.85, 0.9, or 1 mm, or a value within a range between any of the preceding values.

In some embodiments, any of microneedles described herein may further include a microneedle needle base. A microneedle base may be of any shape, but it is typically at least 25% larger than the diameter of the microneedle. A microneedle base may provide stability, but also serve to prevent a microneedle from inserting 100% into the skin surface, thus leaving a desired gap between the skin surface and the wearable medical device. In some embodiments, a microneedle base may be in the shape of frustrum or a conical frustrum.

In many embodiments, any microneedle described herein may be comprised of a plastic, a metal, an absorbable material, or a combination thereof. Suitable plastics include polyolefinic materials, polyesters, polyurethanes, and the like. Suitable metals include stainless steel, titanium, and Nitinol (a nickel/titanium alloy), and the like. Absorbable materials include materials used to form absorbable sutures, such as polyglycolide (e.g., DEXON™), poly (glycolide/lactide) random copolymer (e.g., VICRYL™), and the like

In some embodiments, any microneedle described herein may be coated with one or more electrically conductive substances such that the wearable medical device may serve as a dry electrode.

In some embodiments, any microneedle described herein may be solid or hollow. Hollow microneedles may allow for passage of therapeutics.

In some embodiments, each of the first and second microneedles may be the same. In other embodiments, any one of the first or any one of the second microneedles may differ from one another in one or more aspect described above.

### Applicators

In many embodiments, an applicator for attaching a wearable medical device of the present disclosure to a skin surface is described. The applicator may include a drive actuator configured to drive the first rotationally-distinct segment and the second rotationally-distinct segment in a counter-rotationally manner. For example, applicators having drive actuators may be suited for wearable medical devices described herein having rolling communication members.

In many embodiments, an applicator for attaching a wearable medical device of the present disclosure to a skin surface is described. The applicator may include a loading actuator configured to counter-rotationally load the first rotationally-distinct segment and the second rotationally-distinct segment; a retaining element to retain the wearable medical device in a counter-rotationally loaded configuration; and a mechanism for releasing the wearable medical device from the counter-rotationally loaded configuration. For example, applicators having loading actuators may be suited for wearable medical devices described herein having tensioning communication members.

In some embodiments, the loading actuator may include any combination of mechanical components for effecting rotation of the first and second rotationally-distinct segments. For example, the loading actuator may include one or more of a spring, a gear, a piston, a pump, or the like. In some embodiments, the applicator may include a mechanism for engaging the loading actuator. For example, the mechanism may include twisting the applicator, retracting a plunger, or the like.

In some embodiments, the loading actuator may be tuned to a specific tensioning communication member, or number of communication members. For example, the loading actuator may be configured to counter-rotationally load a wearable medical device at a selected degree of rotation such that the de-tensioning of the communication member is complete or less-than-complete once the wearable medical device is attached to the skin surface. Communication members that have not completely de-tensioned once the wearable medical device is attached to the skin surface may serve to further secure the wearable medical device into the skin surface since the remaining tension will continuously pull the opposing microneedles into the skin. However, too much residual tension in the communication members while in the skin surface may result in injury. Conversely, it is possible overextend a communication member upon application. In other words, a communication member that has de-tensioned past the original configuration may in effect be re-tensioned. An overextended communication member while the wearable medical device is attached to the skin surface may ultimately act to pull the microneedles from the skin surface due to the force favorable to returning to its original configuration, thereby decreasing wear times.

In some embodiments, the applicators described may further include a docking platform for holding a wearable medical device within the applicator.

In some embodiments, the applicators described may further include actuating guides that are configured to mate with applicator guides on the first and second rotationally-distinct segments. Alternatively, the applicator may include a docking platform for holding a wearable medical device within the applicator, and actuating guides configured to mate with applicator guides on a docking platform. In some embodiments, actuating guides may be tracks within stationary walls within the applicator. Tracks within stationary walls of an applicator may be angled to accommodate rotation of the first and/or second rotationally-distinct segment (see, e.g., FIGS. 6A and 6B). Actuating guides may assist independent rotation of the first and second rotationally-distinct segments and thus be of any construction. For example, actuating guides may include tracks, pins, gears, friction-inducing components, or the like.

In some embodiments, a retaining element may include any combination of mechanical components for holding the first and second rotationally-distinct segments in a counter-rotationally loaded configuration. For example, a retaining element may include pins, latches, brackets, or the like.

In some embodiments, a mechanism for releasing the wearable medical device from a counter-rotationally loaded configuration may include any combination of mechanical components to disengage the retaining element. The mechanism may include a button, compressing a plunger, a switch, or the like. Upon disengaging the retaining element, the potential energy stored within the loaded communication member(s) may drive the wearable medical device back to its original state, or at least partly to a de-tensioned state.

In some embodiments, the applicators described may be further configured to remove a wearable medical device from the skin surface. To remove a wearable medical device, an applicator may be contacted to the wearable medical device and the loading actuator engaged effective to rotate the first and second rotationally-distinct segments such that the first microneedles and the second microneedles exit the skin surface.

In some embodiments, the features of the applicators described may be strictly mechanically driven. In other embodiments, the features of the applicators may be at least partly electrically driven.

While the applicators described herein are directed toward aiding attachment of the wearable medical devices of the present disclosure, it is to be understood that said applicators may be useful for attaching wearable medical devices that may depart from the recite scope, so long as said wearable medical devices include a first rotationally-distinct segment, a second rotationally-distinct segment, and a plurality of microneedles.

### Methods for Application

In many embodiments, a method for attaching a wearable medical device to a skin surface is described. The method may include providing a wearable medical device of the present disclosure (e.g., having a tensioning communication member) and rotating the first rotationally-distinct segment and rotating the second rotationally-distinct segment such that the wearable medical device is in a counter-rotationally loaded configuration. The method may include contacting the wearable medical device in the counter-rotationally loaded configuration to a skin surface and releasing the wearable medical device from the counter-rotationally loaded configuration such that the plurality of first microneedles and the plurality of second microneedles are driven into the skin surface. In some embodiments, the rotating of the first rotationally-distinct segment and the rotating the second rotationally-distinct segment may be via mechanical actuators on the wearable medical device. In other embodiments, the rotating of the first rotationally-distinct segment and the rotating the second rotationally-distinct segment may be via an applicator described herein.

In many embodiments, a method for attaching a wearable medical device to a skin surface is described. The method may include providing a wearable medical device of the present disclosure (e.g., having a rolling communication member); contacting the wearable medical device to the skin surface; and rotating the first rotationally-distinct segment and rotating the second rotationally-distinct segment such that the plurality of first microneedles and the plurality of second microneedles are driven into the skin surface. In some embodiments, the rotating of the first rotationally-distinct segment and the rotating the second rotationally-distinct segment may be via mechanical actuators on the wearable medical device. In other embodiments, the rotating of the first rotationally-distinct segment and the rotating the second rotationally-distinct segment may be via an applicator described herein.

In many embodiments, a method for attaching a wearable medical device of the present disclosure (e.g., having a tensioning communication member) to a skin surface is described. The method may include providing an applicator described herein, the applicator having the wearable medical therein; and rotating the first rotationally-distinct segment and rotating the second rotationally-distinct segment such that the wearable medical device is in a counter-rotationally loaded configuration. The method may further include contacting the wearable medical device in the counter-rotationally loaded configuration to the skin surface; and releasing the wearable medical device from the counter-rotationally loaded configuration such that the plurality of first microneedles and the plurality of the second microneedles are driven into the skin surface.

In many embodiments, a method for attaching a wearable medical device to a skin surface is described. The method may include providing an applicator described herein, the applicator having the wearable medical device therein; and rotating a first rotationally-distinct segment and rotating a second rotationally-distinct segment such that the wearable medical device is in a counter-rotationally loaded configuration. The method may further include contacting the wearable medical device in the counter-rotationally loaded configuration to the skin surface; and releasing the wearable medical device from the counter-rotationally loaded configuration such that a plurality of microneedles are driven into the skin surface. The wearable medical device may be any wearable medical device so long as it includes a first rotationally-distinct segment, a second rotationally-distinct segment, and a plurality of microneedles.

In many embodiments, a method for attaching a wearable medical device of the present disclosure (e.g., having a rolling communication member) to a skin surface is described. The method may include providing an applicator described herein, the applicator having the wearable medical device therein; contacting the applicator to the skin surface; and rotating a first rotationally-distinct segment and rotating a second rotationally-distinct segment such that a plurality of microneedles are driven into the skin surface. The wearable medical device may be any wearable medical device so long as it includes a first rotationally-distinct segment, a second rotationally-distinct segment, and a plurality of microneedles.

In many embodiments, a method for attaching a wearable medical device (e.g., having a rolling communication member) to a skin surface is described. The method may include providing an applicator described herein, the applicator having the wearable medical device therein; contacting the applicator to the skin surface; and rotating a first rotationally-distinct segment and rotating a second rotationally-distinct segment such that a plurality of microneedles are driven into the skin surface. The wearable medical device may be any wearable medical device so long as it includes a first rotationally-distinct segment, a second rotationally-distinct segment, and a plurality of microneedles. The wearable medical device may be any wearable medical device so long as it includes a first rotationally-distinct segment, a second rotationally-distinct segment, and a plurality of microneedles.

In some embodiments, any method described herein employing an applicator may further include selecting a degree of rotation for rotating the first rotationally-distinct segment and for rotating the second rotationally-distinct segment, whether it be for loading a wearable medical device (e.g., having a tensioning communication member) or driving a wearable medical device (e.g., having rolling communication member).

In some embodiments, any method described herein for attaching a wearable medical device may further include attaching a monitoring device to the wearable medical device.

In some embodiments, any method described herein for attaching a wearable medical device to skin surface may further include applying a supplementary securement article to the wearable medical device or a wearable medical device having a monitoring device thereon. The supplementary securement article may be a bandage, a protective covering (e.g., water/sweat proof), or the like. In some embodiments, the supplementary securement article may include a backing and a skin-compatible adhesive.

In some embodiments, a method for removing a wearable medical device from a skin surface is described. The method may include engaging mechanical actuators upon the wearable medical device such that the wearable medical device is in a counter-rotationally loaded configuration and lifting the wearable medical device from the skin surface.

In some embodiments, a method for removing a wearable medical device from a skin surface is described. The method may include contacting an applicator to the wearable medical device on the skin surface, engaging a loading actuator within the applicator such that the wearable medical device is in a counter-rotationally loaded configuration, and lifting the wearable medical device from the skin surface.

### Methods for Monitoring

In many embodiments, a method for monitoring a biological signal is described. The method may include detecting a biological signal with the monitoring device secured to a wearable medical device of the present disclosure that is attached to a skin surface.

In some embodiments, the biological signal may be selected from an electrical signal, a chemical signal, a light remittance signal, or a combination thereof.

The method may further include attaching a wearable medical device to a skin surface.

The method may further include securing a monitoring device to a wearable medical device attached to the skin surface.

### Kits

In many embodiments, a kit is described. The kit may include a wearable medical device of the present disclosure and a set of instructions for attaching the wearable medical device to a skin surface. In some embodiments, the kit may further include one or more of: an applicator and a monitoring device.

In many embodiments, a kit is described. The kit may include a wearable medical device of the present disclosure, an applicator described herein, and a set of instructions for attaching the wearable medical device to a skin surface. In some embodiments, the kit may further include a monitoring device.

In some embodiments, any kit described herein may further include one or more supplementary securement article.

1. A wearable medical device comprising:
a base comprising:
a first rotationally-distinct segment,
a second rotationally-distinct segment, the second rotationally-distinct segment at least partly surrounding the first rotationally-distinct segment,
a plurality of first microneedles located on the first rotationally-distinct segment, and
a plurality of second microneedles located on the second rotationally-distinct segment; and
at least one communication member, the at least one communication member being in communication with the first rotationally-distinct segment and the second rotationally-distinct segment.

2. (canceled)

3. The wearable medical device of claim 1, the base further comprising a first mechanical actuator in communication with the first rotationally-distinct segment and a second mechanical actuator in communication with the second rotationally-distinct segment; or
wherein each of the first rotationally-distinct segment and the second rotationally-distinct segment include one or more applicator guide.

4. The wearable medical device of claim 1, the base further comprising a flexible membrane extending beyond a base periphery, the flexible membrane comprising an adhesive, and optionally the flexible membrane comprising one or more applicator guide thereon.

5. (canceled)

6. The wearable medical device of claim 1, wherein the first rotationally-distinct segment and the second rotationally-distinct segment are each in a shape of a cylindrical ring and are arranged in a concentric manner.

7. (canceled)

8. The wearable medical device of claim 1, wherein at least a portion of the plurality of first microneedles and at least a portion of the plurality of second microneedles are independently characterized by an elevation angle of about 40° to 80°.

9. The wearable medical device of claim 1, wherein each first microneedle that is characterized by an elevation angle of about 40° to 80° is oriented such that first microneedle tips face one rotational direction, and each second microneedle that is characterized by an elevation angle of about 40° to 80° is oriented such that second microneedle tips face a rotational direction opposite to that of the first microneedle tips.

10. The wearable medical device of claim 1, wherein at least a portion of the plurality of first microneedles and at least a portion of the plurality of second microneedles are independently characterized by an elevation angle of about 40° to 80° and independently arranged at an orientation angle of −25° to 25°.

11. The wearable medical device of claim 1, wherein the plurality of first microneedles and the plurality of second microneedles are each independently characterized by a length of about 0.2 mm to about 3.0 mm.

12. The wearable medical device of claim 1, wherein the plurality of first microneedles and the plurality of second microneedles are each independently characterized by a diameter of about 1 μm to about 25 μm.

13. The wearable medical device of claim 1, wherein one or more of:
at least a portion of the plurality of first microneedles or at least a portion of the plurality of second microneedles are characterized by a nonuniform diameter, and
a least a portion of the plurality of first microneedles or at least a portion of the plurality of second microneedles are barbed.

14. (canceled)

15. The wearable medical device of claim 1, the plurality of first microneedles arranged in a at least one row and the plurality of the second microneedles arranged in at least one row.

16. The wearable medical device of claim 1, the at least one communication member selected from a flexible rod, a flexible band, and a spring.

17. The wearable medical device of a claim 1, the at least one communication member being a rolling disc.

18. An applicator for attaching a wearable medical device of claim 1 to a skin surface,
the applicator comprising:
a loading actuator configured to counter-rotationally load the first rotationally-distinct segment and the second rotationally-distinct segment;
a retaining element to retain the wearable medical device in a counter-rotationally loaded configuration; and
a mechanism for releasing the wearable medical device from the counter-rotationally loaded configuration; or
the applicator comprising:
a drive actuator configured to rotate a first rotationally-distinct segment and rotate a second rotationally-distinct segment in a counter-rotational manner.

19. (canceled)

20. A method for attaching a wearable medical device to a skin surface, the method comprising:
providing a wearable medical device of claim 1;
rotating the first rotationally-distinct segment and rotating the second rotationally-distinct segment such that the wearable medical device in a counter-rotationally loaded configuration;
contacting the wearable medical device in the counter-rotationally loaded configuration to the skin surface; and
releasing the wearable medical device from the counter-rotationally loaded configuration such that the plurality of first microneedles and the plurality of second microneedles are driven into the skin surface.

21. A method for attaching a wearable medical device to a skin surface, the method comprising:
providing a wearable medical device of claim 1;
contacting the wearable medical device to the skin surface; and
rotating the first rotationally-distinct segment and rotating the second rotationally-distinct segment such that the plurality of first microneedles and the plurality of second microneedles are driven into the skin surface.

22. A method for attaching a wearable medical device to a skin surface, the method comprising:
providing an applicator of claim 18, the applicator having the wearable medical device therein;
rotating the first rotationally-distinct segment and rotating the second rotationally-distinct segment such that the wearable medical device is in a counter-rotationally loaded configuration;
contacting the wearable medical device in the counter-rotationally loaded configuration to the skin surface; and
releasing the wearable medical device from the counter-rotationally loaded configuration such that the plurality of first microneedles and the plurality of second microneedles are driven into the skin surface.

23. (canceled)

24. A method for monitoring a biological signal, the method comprising:
detecting a biological signal with a monitoring device that is secured to a wearable medical device of claim 1, the wearable medical device being attached to a skin surface.

25. A kit comprising:
a wearable medical device of claim 1;
a set of instructions directing a user to attach the wearable medical device to a skin surface.

26. The kit of claim 25, further comprising one or more of:
an applicator, and
a monitoring device.
